(19) United States
(12) Patent Application Publication
(10) Pub. No.: US 2018/0339245 A1
(43) Pub. Date: Nov. 29, 2018

(54) DEVICE AND METHOD FOR DETECTING POLLUTANTS IN WATER

(71) Applicant: NATIONAL PINGTUNG UNIVERSITY OF SCIENCE & TECHNOLOGY, Pingtung County (TW)

(72) Inventors: Sheng-Chi Lin, Pingtung County (TW); Tsun-Kuo Chang, Taipei (TW); Wei-Jhan Syu, Taichung (TW); Yen-Shan Huang, Pingtung County (TW); Cheng-Hsun Lee, Taipei (TW); Shao-Yu Peng, Pingtung County (TW); Yu-Ting Wu, Pingtung County (TW)

(21) Appl. No.: 15/850,581

(22) Filed: Dec. 21, 2017

(30) Foreign Application Priority Data
May 23, 2017 (TW) 106117016

(51) Int. Cl. B01D 15/36; G01N 33/18; G01N 21/64

(52) U.S. Cl. CPC B01D 15/361; G01N 21/64; G01N 33/182; G01N 33/1813

(57) ABSTRACT

A device for detecting pollutants in water includes a housing and an ion exchange layer. A detecting face is formed on an outer face of the housing. The housing has an injection port and a discharge port opposite to the injection port. The injection port is coupled with a liquid disperser. The ion exchange layer is disposed between the liquid disperser and the discharge port. The ion exchange layer is aligned with the detecting face of the housing. Therefore, the pollutants have even concentrations all over the ion exchange layer to avoid the uneven distributions of the pollutants. This provides an advantage of improved detection accuracy. Besides, the device can further analyze detailed information of the pollutants based on the fluorescent signals generated by the gradients of the concentrations.

### CROSS REFERENCE TO RELATED APPLICATIONS

The application claims the benefit of Taiwan application serial No. 106117016, filed on May 23, 2017, the subject matter of which is incorporated herein by reference.

### BACKGROUND OF THE INVENTION

### 1. Field of the Invention

The present invention generally relates to a device for detecting pollutants in water and a method for detecting pollutants in water and, more particularly, to a device and a method for detecting pollutants in water which allow pollutants in water to be distributed evenly.

### 2. Description of the Related Art

Ion exchange system can exchange and remove ions, concentrate and recycle, and can easily recycle pollutants such as heavy metals, etc. Therefore, it is widely used for detecting waste water. The conventional method for detecting waste water by ion exchange system is carried by mixing a waste water sample and an ion exchange resin in order to allow the ion exchange resin to absorb pollutants in the waste water sample. Subsequently, an analytical machine such as X-ray fluorescence (XRF) can be used to emit X-ray on the ion exchange resin to excite the pollutants to generate fluorescent radiations. The fluorescent radiations can be received and analyzed by the XRF to detect the pollutants in the waste water sample.

However, when mixing the waste water sample and the ion exchange resin, the waste water sample is usually directly poured into the ion exchange resin and will mix heterogeneously with the ion exchange resin. This will cause uneven distribution of the pollutants in the ion exchange resin that may mislead detection result since the X-ray emitted from the XRF may irradiate on areas with less pollutants absorbed or on areas with more pollutants absorbed. Therefore, the detection result obtained by the conventional method will be inaccurate.

In light of this, it is necessary to improve the accuracy of detecting pollutant in water.

### SUMMARY OF THE INVENTION

It is therefore the objective of this invention to provide a device for detecting pollutants in water and a method for detecting pollutants in water that allow the pollutants in water to be distributed evenly in an ion exchange resin to improve detection accuracy.

When the terms “inner”, “outer” and similar terms are used herein, it should be understood that these terms have reference only to the structure shown in the drawings as it would appear to a person viewing the drawings and are utilized only to facilitate describing the invention, rather than restricting the invention.

In an embodiment of the invention, a device for detecting pollutants in water is disclosed, which includes: a housing having an injection port, a discharge port opposite to the injection port, and a detecting face formed on an outer face of the housing, with the injection port being coupled with a liquid disperser, and an ion exchange layer disposed between the liquid disperser and the discharge port, and aligned with the detecting face of the housing.

Accordingly, the device for detecting pollutants in water of the present invention allows the waste water sample to flow through a plurality of through holes evenly disposed on the liquid disperser. Consequently, the pollutants in the waste water sample can be distributed and enter into the ion exchange layer evenly. As such, the pollutants in the waste water sample can be absorbed by ion exchange resin particles in the ion exchange layer in order. Therefore, the pollutants in the ion exchange layer can be distributed evenly to avoid inaccurate detection result caused by uneven distribution of the pollutants due to the visible light or the ultraviolet light emitted from an analytical machine irradiating on areas with more or with less pollutants absorbed. This provides an advantage of improved detection accuracy. Besides, the device can further analyze detailed information of the pollutants based on fluorescent signals generated by the concentration gradients of the pollutants in the ion exchange layer.

In an example, the detecting face includes a flat face. As such, the device can receive the fluorescent signals from the detecting face evenly. Therefore, the accuracy of the detection can be improved.

In an example, the ion exchange layer is filled with a plurality of ion exchange resin particles. As such, proper ion exchange resin particles can be selected according to the pollutants to be absorbed.

In an example, the discharge port has a diameter smaller than a particle size of each of the plurality of ion exchange resin particles. As such, it can prevent the ion exchange resin particle from dropping out from the ion exchange layer.

In an example, the device further includes a stopper. The stopper is disposed between the ion exchange layer and the discharge port. The stopper has a plurality of passing holes. Each of the plurality of passing holes has a diameter smaller than the particle size of each of the plurality of ion exchange resin particles. As such, it can prevent the ion exchange resin particle from dropping out from the ion exchange layer.

In another embodiment of the invention, a method for detecting pollutants in water by using the aforementioned device is disclosed, which includes: injecting a waste water sample into the injection port, allowing the waste water sample flow through the liquid disperser to make the waste water sample distributed evenly in the ion exchange layer, and emitting a visible light or an ultraviolet light to the ion exchange layer to analyze the concentration gradients of the pollutants in the waste water sample.

Accordingly, the method for detecting pollutants in water of the present invention is based on making the waste water sample flow through the plurality of through holes evenly disposed on the liquid disperser. Consequently, the waste water sample can be dispersed and enter into the ion exchange layer evenly. As such, the pollutants in the waste water sample can be absorbed by the ion exchange resin particles in the ion exchange layer in order and the pollutants can be distributed evenly in the ion exchange layer. The method can avoid inaccurate detection results caused by uneven distributions of the pollutants due to the visible light or the ultraviolet light emitted from the analytical machine irradiating on areas with more or with less pollutants absorbed. This provides an advantage of improved detection accuracy. Besides, the method can further analyze detailed information of the pollutants based on fluorescent signals generated by the concentration gradients of the pollutants in the ion exchange layer.

In an example, the visible light or the ultraviolet light can irradiate the ion exchange layer from the detecting face. Therefore, the detecting device can receive the fluorescent signals transmitted from the detecting face evenly to improve detection accuracy. Besides, according to different analytical machines and different detection requirements, the detecting face of the device provides steady support when the device is placed on the analytical machine. This also provides an advantage of improved convenience.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is an exploded and perspective view of the first embodiment of the device for detecting pollutants in water according to the present invention.

FIG. 2 is a cross sectional view of the first embodiment of the device for detecting pollutants in water according to the present invention.

FIG. 3 is a cross sectional view of the second embodiment of the device for detecting pollutants in water according to the present invention.

FIG. 4 shows the detection results of using the device for detecting pollutants in water according to the present invention.

### DETAILED DESCRIPTION OF THE INVENTION

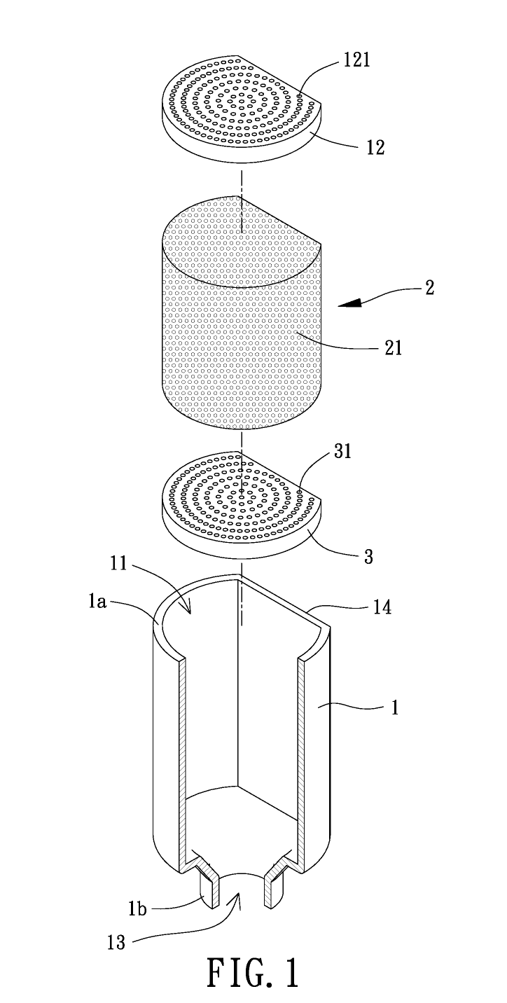

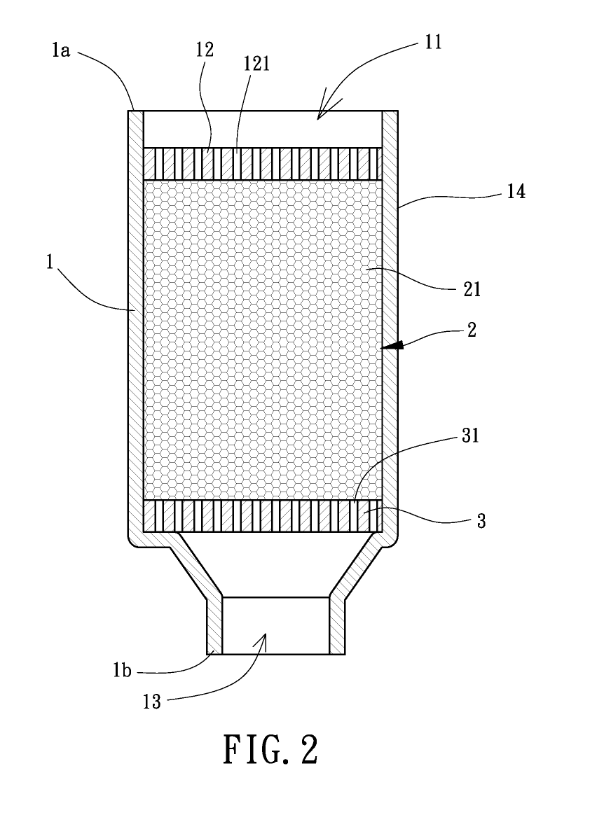

With reference to FIGS. 1 and 2, the first embodiment of the device for detecting pollutants in water of the present invention includes a housing 1 and an ion exchange layer 2 received in the housing 1. A waste water sample can be injected into the housing 1 to allow the waste water sample to flow through the ion exchange layer 2.

The housing 1 can have a noncircular cross-section to prevent the housing 1 from rolling when it is tipped. The material of the housing 1 can be selected from materials that allow a visible light or an ultraviolet light to pass through. Therefore, a visible light or an ultraviolet light emitted from an analytical machine can pass through the housing 1 to irradiate on the pollutants in the device for detecting pollutants in water. Besides, a material of the housing 1 is preferably selected from materials that do not contain the pollutants to be detected. For example, when the pollutants to be analyzed in the waste water example are heavy metals, preferably, the housing 1 comprises non-metal material to avoid affecting the detection results.

The housing 1 has a first end 1a and a second end 1b opposite to the first end 1a. When the waste water sample is injected into the housing 1, it can flow from the first end 1a of the housing 1 to the second end 1b of the housing 1. More specifically, the first end 1a has an injection port 11 and the waste water sample can be injected into the housing 1 via the injection port 11. The housing 1 can be coupled with a liquid disperser 12 at the injection port 11 to allow the waste water sample injected from injection port 11 to be distributed evenly in the housing 1 after flowing through the liquid disperser 12. The liquid disperser 12 can be fixed at an inner wall of the injection port by means of engaging, tightly matching, or screwing. However, these are merely exemplary and are not limitative of the invention. Besides, a shape of the liquid disperser 12 can preferably be varied according to a shape of a cross-section of the injection port 11. As such, the liquid disperser 12 can block in between the injection port 11 and the housing 1 completely. Therefore, all of the waste water sample is allowed to flow through the liquid disperser 12 to ensure the evenly distribution of the pollutants.

More specifically, the liquid disperser 12 has a plurality of through holes 121 that penetrates through two opposite surfaces thereof, and the plurality of through holes 121 is disposed evenly on the two opposite surfaces of the liquid disperser 12. For example, the plurality of through holes 121 can be radial from a geometric center to a surrounding of the liquid disperser 12, or be arranged as concentric circles centered at the geometric center of the liquid disperser 12, or in other geometric alignments. However, these are merely exemplary and are not limitative of the invention. It is worth noting that, the plurality of through holes 121 on the liquid disperser 12 can have certain distance among each other to serve as a buffer area. Preferably, the distance among each of the plurality of through holes 121 is two times to the diameter of the plurality of through holes 121. Therefore, the flow rate of the waste water sample will not be too fast while flowing through the liquid disperser 12. In other words, when the waste water sample reaches the liquid disperser 12, it will not directly flow into the housing 1 from the plurality of through holes 121 where it contacts. As such, the rest of the waste water sample can flow to the plurality of through holes 121 at other areas smoothly to avoid affecting the evenly distribution of the waste water sample. Therefore, the injected waste water sample can be distributed evenly in the housing 1 by flowing through the plurality of the through holes 121 rather than just being injected from a single inlet. This provides an advantage to make the pollutants in the waste water sample be injected and distributed evenly in the housing 1. Besides, the waste water sample injected into the housing 1 can be drained away from a discharge port 13 on the second end 1b of the housing 1.

The outer face of the housing 1 has a detecting face 14. The detecting face 14 allows the aforementioned visible light or ultraviolet light to pass through in order to detect the waste water sample. In the embodiment, the detecting face 14 can have a flat face. Therefore, the analytical machine can receive the fluorescent signals transmitted from the detecting face 14 evenly to improve the detection accuracy. Besides, according to different analytical machines and different detection requirements, the detecting face 14 of the device provides steady support when the device is placed on the analytical machine. This also provides an advantage of improved convenience.

The ion exchange layer 2 can be disposed between the liquid disperser 12 and the discharge port 13, and is aligned with the detecting face 14. As such, the waste water sample flew through the liquid disperser 12 can further flow through the ion exchange layer 2 and then be drained away from the discharge port 13. Therefore, the pollutants in the waste water sample can be absorbed in the ion exchange layer 2 for detecting. More specifically, ion exchange resin particles 21, which are used as substance, can be filled to form the ion exchange layer 2. The ingredients of the ion exchange resin particles 21, which are readily appreciated by the skilled persons, can be styrene, acrylic acid or polymerized by other monomers. Besides, proper materials of the ion exchange resin particles 21 can be selected according to the pollutants to be absorbed. For example, it can be cation-exchange resin in order to absorb positively-charged pollutants, or it can be anion-exchange resin in order to absorb negatively-charged pollutants, or it can be chelating resin that has affinity and selectivity toward heavy metals in order to analyze heavy metal pollutants. However, these are merely exemplary and are not limitative of the invention.

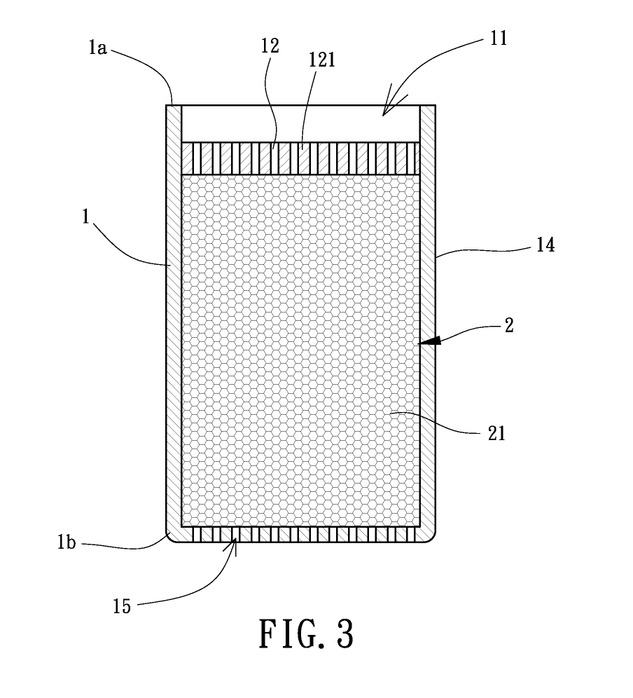

Besides, in order to prevent the ion exchange resin particles 21 in the ion exchange layer 2 from dropping out from the discharge port 13 of the housing 1, a stopper 3 can be disposed in between the ion exchange layer 2 and the discharge port 13. The stopper 3 can be fixed at the inner wall of the discharge port 13 by means of engaging, tightly matching, or screwing. However, these are merely exemplary and are not limitative of the invention. The stopper 3 can have a plurality of passing holes 31, and each of the plurality of passing holes 31 has a diameter smaller than the particle size of the ion exchange resin particles 21. Besides, the stopper 3 can also has a mesh with apertures being smaller than the particle size of the ion exchange resin particles 21. As such, the waste water sample can flow through it and be drained away from the discharge port 13 without having the ion exchange resin particles 21 dropping out from the discharge port 13. With reference to FIG. 3, the second end 1b of the housing 1 can have a plurality of discharge ports 15. Each of the plurality of discharge port 15 has a diameter smaller than particle size of the ion exchange resin particles 21. Therefore, it provides an advantage of preventing ion exchange resin particles 21 from dropping out from the ion exchange layer 2.

According to the aforementioned structure, the device for detecting pollutants in water of the present invention can perform a method for detecting pollutants in water. The method includes injecting a waste water sample into the injection port 11 and allowing the waste water sample to flow through the liquid disperser 12 to make the waste water sample flow through the plurality of through holes 121 evenly disposed on the liquid disperser 12. Then, allowing the waste water sample to be distributed evenly in the ion exchange layer 2 and to be drained away from the discharge port 13. In the meantime, the pollutants in the waste water sample are evenly absorbed in order by the plurality of ion exchange resin particles 21 starting from the ones adjacent to the liquid disperser 12. Afterwards, emitting a visible light or an ultraviolet light from the analytical machine to pass through the detecting face 14 of the housing 1 in order to excite the pollutants in the ion exchange layer 2, which is aligned with the detecting face 14, to generate fluorescent signals. The fluorescent signals are received and analyzed by the analytical machine.

Besides, the pollutants will form a concentration gradient in the ion exchange layer 2 due to the influence of absorption efficiency. As such, different kinds of pollutants will form different concentration gradients in the ion exchange layer 2. Therefore, with reference to a database, detailed information of the pollutants can be obtained by analyzing the fluorescent signals generated by the concentration gradients. For example, checking points can be formed from the concentration gradients to detect respective fluorescent signals thereof. Moreover, referring to the database varying from the pollutants to be analyzed, proper checking points can be selected as the average concentrations of the pollutants in the whole waste water sample. As such, the concentrations of the pollutants in the waste water sample can be obtained by calculating the average concentrations.

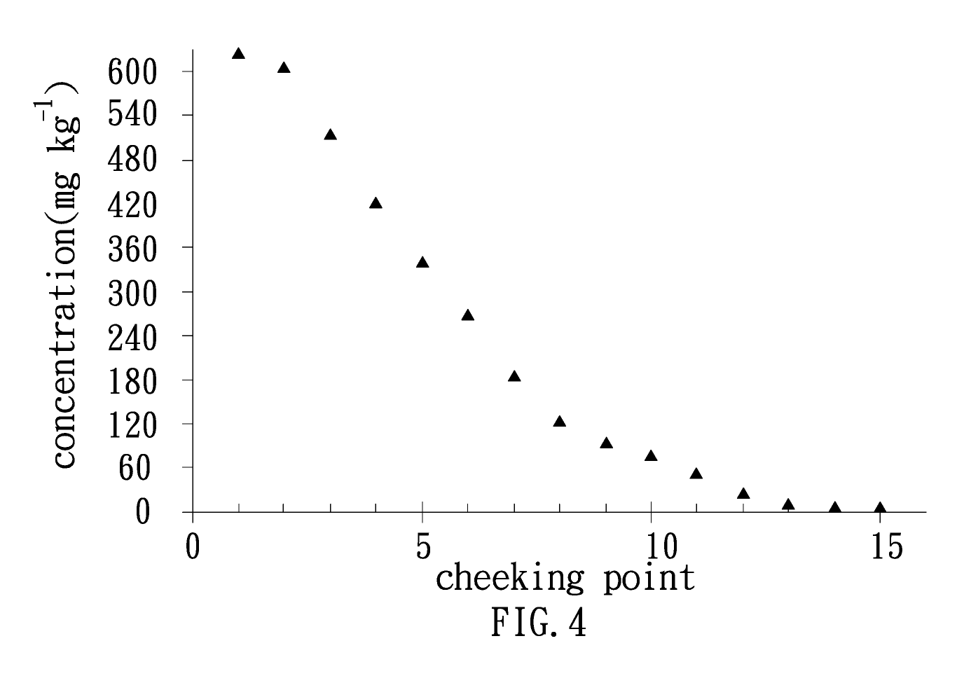

For example, in the embodiment, 10 g of the ion exchange resin particles 21 were filled in the device for detecting pollutants in water to form the ion exchange layer 2. Then, injecting 250 mL of 10 mg L⁻¹ heavy metal copper into the ion exchange layer 2 to form a concentration gradient of the heavy metal cooper. Afterwards, the heavy metal copper in the ion exchange layer 2, which is aligned with the detecting face 14, is excited by the analytical machine to generate fluorescent signals. The fluorescent signals are received and analyzed by the analytical machine. Consequently, 15 checking points, which are formed in the ion exchange layer 2 starting from an end adjacent to the first end 1a, to an end adjacent to the second end 1b, are excited to generate fluorescent signals and the fluorescent signals are received. The detection results are shown in FIG. 4 and Table 1.

TABLE 1
The concentrations of heavy metal in each check point

| Checking Points | Concentration (mg/kg) |
|---|---|
| 1 | 627.4 |
| 2 | 607.9 |
| 3 | 515.3 |
| 4 | 423.7 |
| 5 | 340.3 |
| 6 | 250.6 |
| 7 | 187.1 |
| 8 | 124.2 |
| 9 | 95.9 |
| 10 | 77.6 |
| 11 | 54.2 |
| 12 | 25.5 |
| 13 | 12.0 |
| 14 | 10.3 |
| 15 | 6.4 |

After referring to the database, it can be known that the sixth checking point is the average concentration, which is 250.6 mg kg⁻¹, of the heavy metal copper absorbed in the ion exchange layer 2. Therefore, the overall weight of the copper absorbed in the ion exchange layer 2 can be calculated by the following equation: 10 (g)*10⁻³ (kg g⁻¹)*250.6 (mg kg⁻¹)=2.506 (mg). Assuming the average absorption efficiency is 95%, the following equation can be obtained based on material balance: X (mg L⁻¹)*250 (mL)*10⁻³ (L mL⁻¹)*95%=2.506 (mg). It can be calculated that X, the concentration of the heavy metal in the water, is 10.55 mg L⁻¹. Compared with the actual concentration of the injected heavy metal copper, the error of the detection is only 5.5%. Therefore, the device for detecting pollutants in water of the present invention can indeed detect the pollutants in the waste water sample.

In view of the foregoing, the device for detecting pollutants in water and the method thereof of the present invention allow the waste water sample to flow through the plurality of through holes evenly disposed on the liquid disperser. The waste water sample can be dispersed and can flow into the ion exchange layer evenly to allow the pollutants in the waste water sample to be absorbed by the ion exchange resin particles in the ion exchange layer in order. As such, the pollutants in the ion exchange layer can be distributed evenly to avoid inaccurate detection result causing by uneven distribution of the pollutants due to the visible light or the ultraviolet light emitted from the analytical machine irradiating on areas with more or with less pollutants absorbed. This provides an advantage of improved detection accuracy. Besides, the device can further analyze detailed information of the pollutants based on the fluorescent signals generated by the concentration gradients of the pollutants in the ion exchange layer.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A device for detecting pollutants in water, comprising:
a housing having an injection port, a discharge port opposite to the injection port, and a detecting face formed on an outer face of the housing, wherein the injection port is coupled with a liquid disperser; and
an ion exchange layer disposed between the liquid disperser and the discharge port, and aligned with the detecting face of the housing.

2. The device for detecting pollutants in water according to claim 1, wherein the detecting face comprises a flat face.

3. The device for detecting pollutants in water according to claim 1, wherein the ion exchange layer is filled with a plurality of ion exchange resin particles.

4. The device for detecting pollutants in water according to claim 1, wherein the discharge port has a diameter smaller than a particle size of each of the plurality of ion exchange resin particles.

5. The device for detecting pollutants in water according to claim 3, wherein the device further comprises a stopper disposed between the ion exchange layer and the discharge port, the stopper comprises a plurality of passing holes, and each of the plurality of passing holes has a diameter smaller than a particle size of each of the plurality of ion exchange resin particles.

6. The device for detecting pollutants in water according to claim 4, wherein the device further comprises a stopper disposed between the ion exchange layer and the discharge port, the stopper comprises a plurality of passing holes, and each of the plurality of passing holes has a diameter smaller than the particle size of each of the plurality of ion exchange resin particles.

7. A method for detecting pollutants in water using the device for detecting pollutants in water according to claim 1, comprising:
injecting a waste water sample into the injection port;
allowing the waste water sample to flow through the liquid disperser to make the waste water sample distributed evenly in the ion exchange layer; and
emitting a visible light or an ultraviolet light to the ion exchange layer to analyze the pollutants in the waste water sample.

8. The method for detecting pollutants in water according to claim 7, wherein emitting the visible light or the ultraviolet light comprises emitting the visible light or the ultraviolet light to the ion exchange layer from the detecting face.
